(19) United States
(12) Patent Application Publication
(10) Pub. No.: US 2014/0243719 A1
(43) Pub. Date: Aug. 28, 2014

(54) Neonatal splint

(71) Applicants: Dominique Ruel, Levis (CA); Suzanne Belisle, Chibougamau (CA)

(72) Inventors: Dominique Ruel, Levis (CA); Suzanne Belisle, Chibougamau (CA)

(21) Appl. No.: 13/776,762

(22) Filed: Feb. 26, 2013

(51) Int. Cl. A61F 5/058

(52) U.S. Cl. CPC A61F 5/058
USPC 602/5

(57) ABSTRACT

A Neonatal set of splints has a set of splints wherein each one is shaped according to the member to be immobilized: A hand splint, an arm splint, an external right foot or internal left foot splint, an internal right foot or external left foot splint.

### FIELD OF THE INVENTION

The present invention relates generally to medical devices but more particularly to a splint for new born.

### BACKGROUND OF THE INVENTION

There are many types of splints for use depending on the type of injury. However, those splints are usually for adults who need to have one or more members immobilized during the healing process.

### SUMMARY OF THE INVENTION

In view of the foregoing disadvantages inherent in the known devices now present in the prior art, the present invention, which will be described subsequently in greater detail, is to provide objects and advantages which are:

To provide for a splint for new born babies not so much for reason of injury but rather to keep them from moving while performing certain care, such as performing an intravenous perfusion on a newborn.

In order to do so, the invention is comprised of a set of neonatal splints comprising a hand splint, an arm splint, and a foot splint. The hand splint including a rectangular portion having two perimeter portions located at opposite end portions thereof, and a tear drop shaped portion extending integrally from a middle side portion thereof in between the two perimeter portions. The arm splint having a rectangular shape with two perimeter portions located at opposite end portions thereof, and the foot splint including two rectangular portions connected together at adjacent end portions, such that the rectangular portions form an angle to one another.

The set of splints has the hand splint further include an elongated reinforcing spine member in between and parallel to the two perimeter portions and extending onto a middle section of the tear drop portion.

The arm splint further includes an elongated reinforcing spine member in between and parallel to the two perimeter portions.

One rectangular portion of the two rectangular portions of the foot splint is longer than a second of the two rectangular portions of the foot splint, and wherein the longer rectangular portion further includes an elongated reinforcing spine member along a length thereof.

Each of the two rectangular portions of the foot splint has two perimeter portions located at opposite side portions thereof.

The set of splints comprises two of the hand splints, two of the arm splints, and two of the foot splints. The rectangular portions of one of the two foot splints are connected together at an angle to one another such that the two foot splints form a mirror-image of one another and thereby form a right foot splint and a left foot splint.

Each of the perimeter portions are formed from a flexible plastic material, thereby being bendable and formable, such that the respective perimeter portions are adapted to be pressed against a portion of a respective hand, arm, or leg and retain their shape.

Each respective spine member is padded on an interior surface thereof.

There has thus been outlined, rather broadly, the more important features of the invention in order that the detailed description thereof that follows may be better understood, and in order that the present contribution to the art may be better appreciated. There are additional features of the invention that will be described hereinafter and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

These together with other objects of the invention, along with the various features of novelty which characterize the invention, are pointed out with particularity in the claims annexed to and forming a part of this disclosure. For a better understanding of the invention, its operating advantages and the specific objects attained by its uses, reference should be made to the accompanying drawings and descriptive matter which contains illustrated preferred embodiments of the invention.

### BRIEF DESCRIPTION OF THE DRAWINGS

FIG. 1 Plan view of the invention in context.

FIGS. 2a-b Isometric views of the hand splint and its underside.

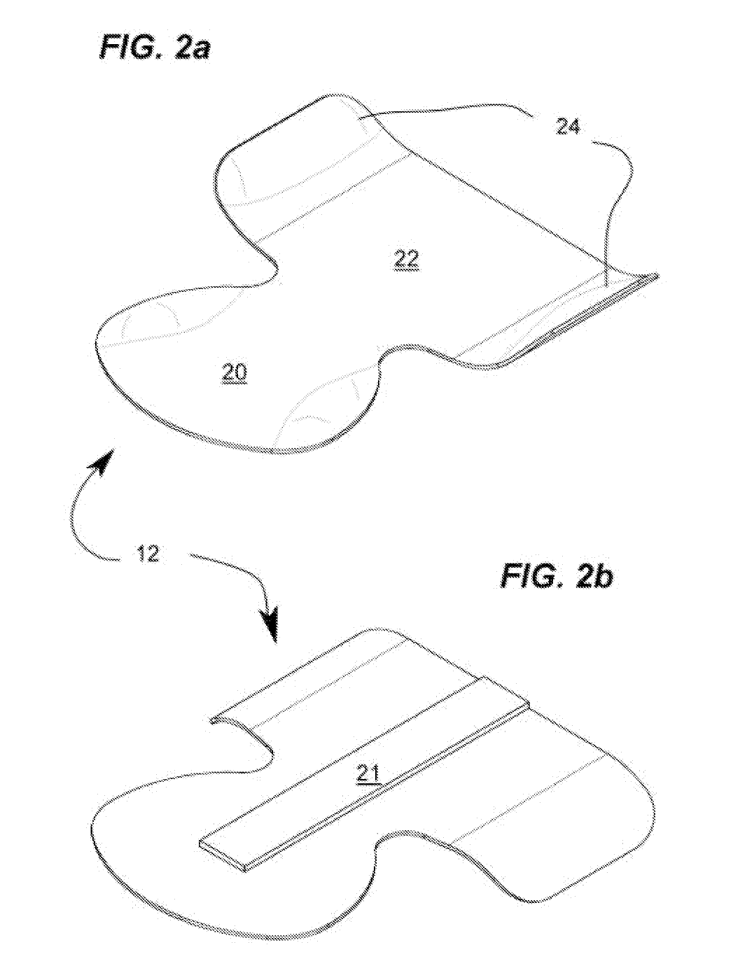

FIGS. 3a-b Isometric views of the arm splint and its underside.

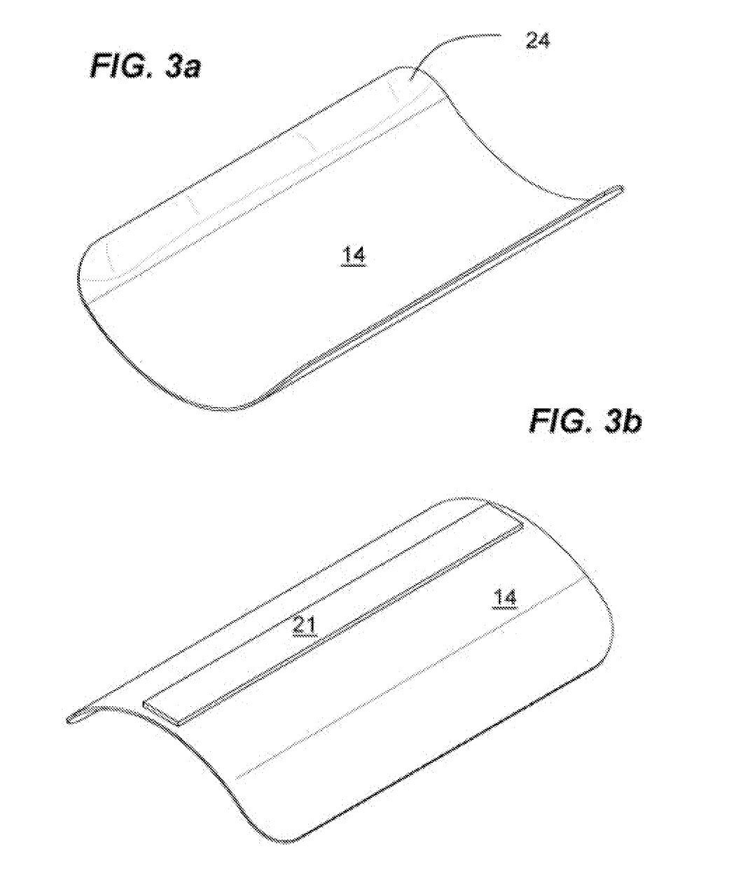

FIGS. 4a-b Isometric views of the foot splint and its underside.

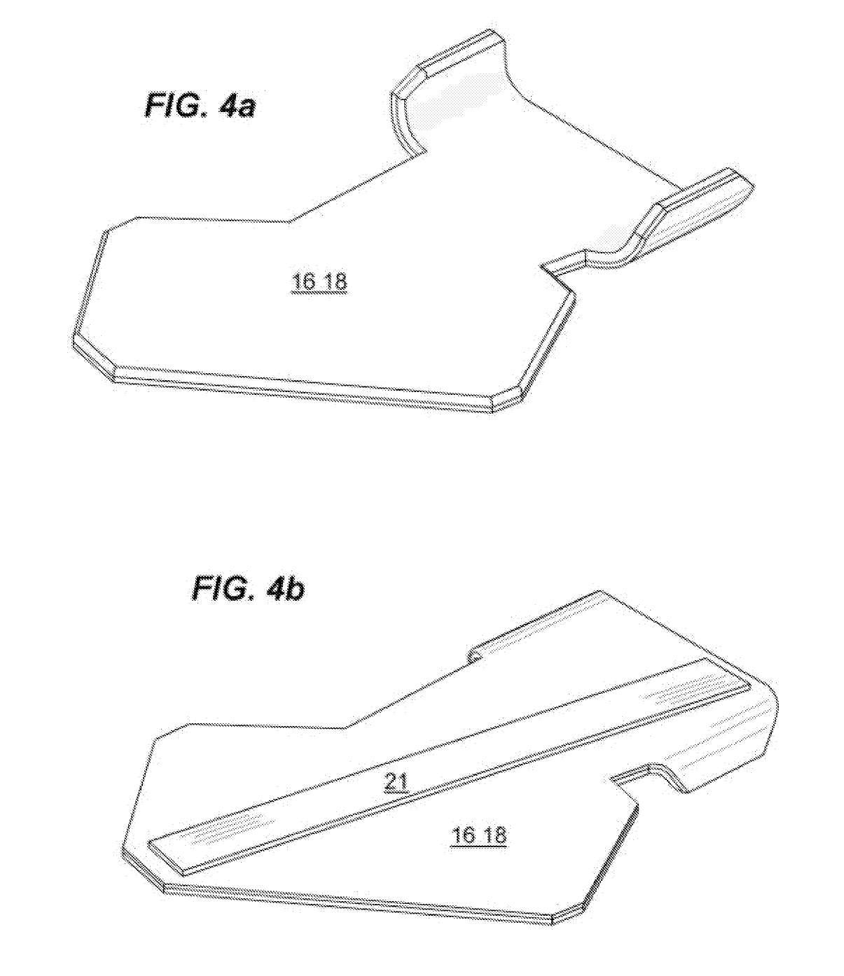

FIGS. 5a-b Plan views of the hand and arm splints with bendable perimeters.

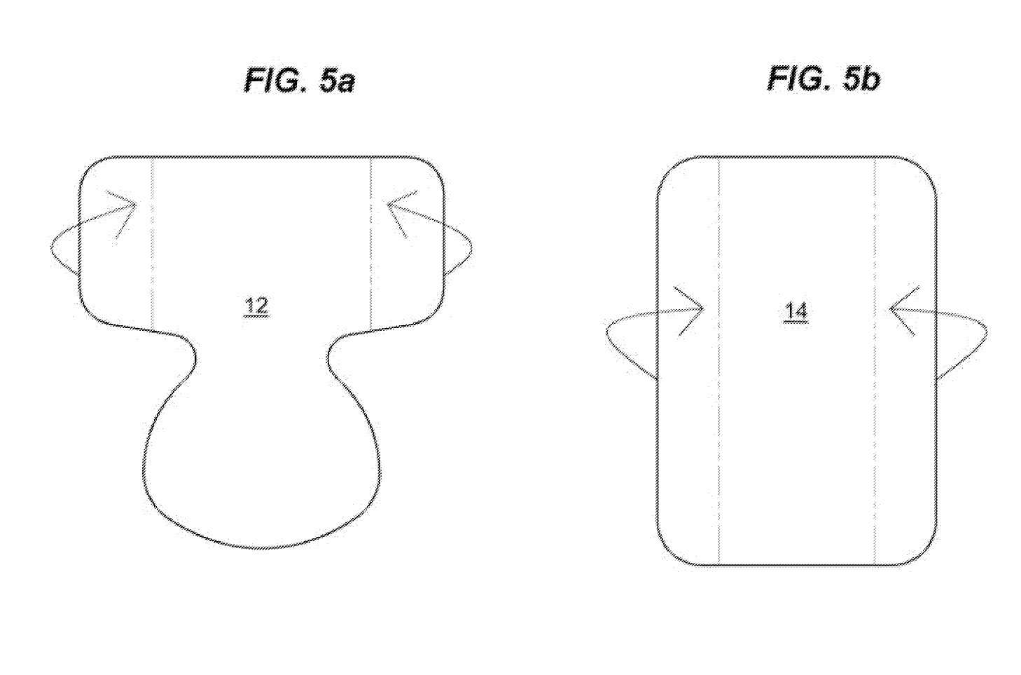

FIG. 6 Plan view of the left and right foot splints.

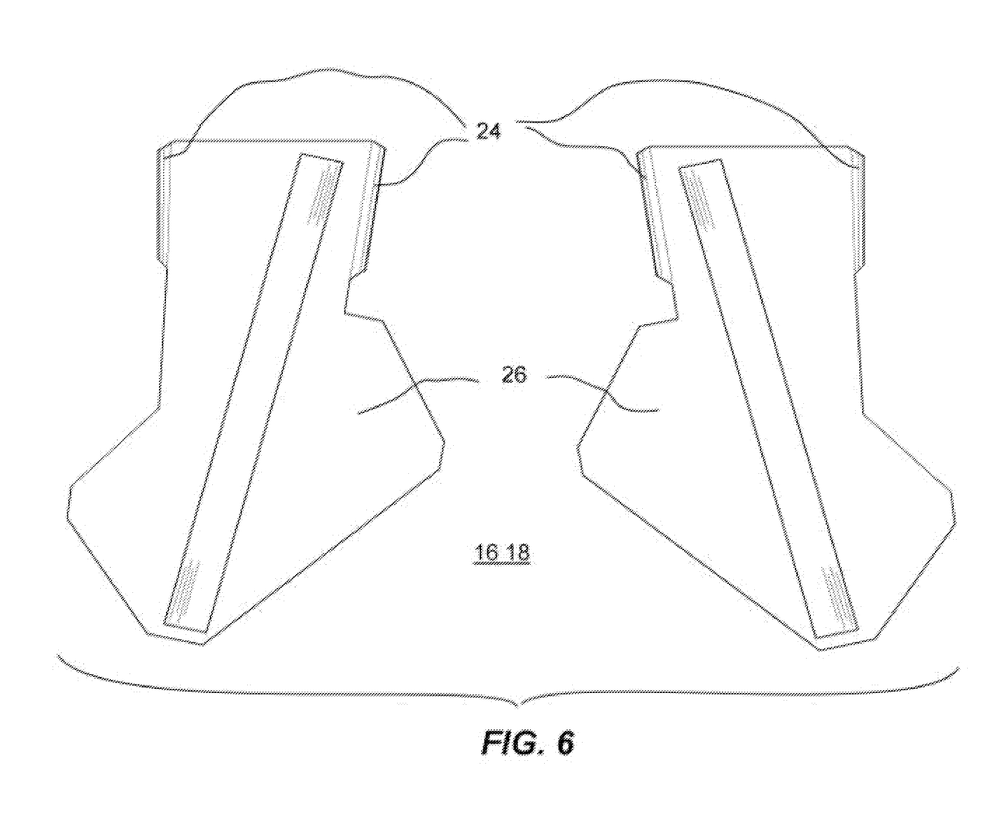

### DETAILED DESCRIPTION OF THE PREFERRED EMBODIMENT

A Neonatal set of splints (10) wherein, each one is shaped according to the member to be immobilized. The splints are made of lightweight, flexible, plastic suitable for immobilizing the desired small members. A spine (21) provides rigidity along the longitudinal axis of the splints (10).

A first splint is a hand splint (12). Another is an arm splint (14); an external right foot or internal left foot splint (16); an internal right foot or external left foot splint (18).

The hand splint (12) has a teardrop portion (20) extending integrally from a rectangular portion (22). Both portions (20, 22) have bendable curved perimeters (24).

The arm splint (14) is rectangular and has its bendable curved perimeter (24) running along its length.

Both foot splints (16, 18) have a bend (26) and bendable curved perimeters (24).

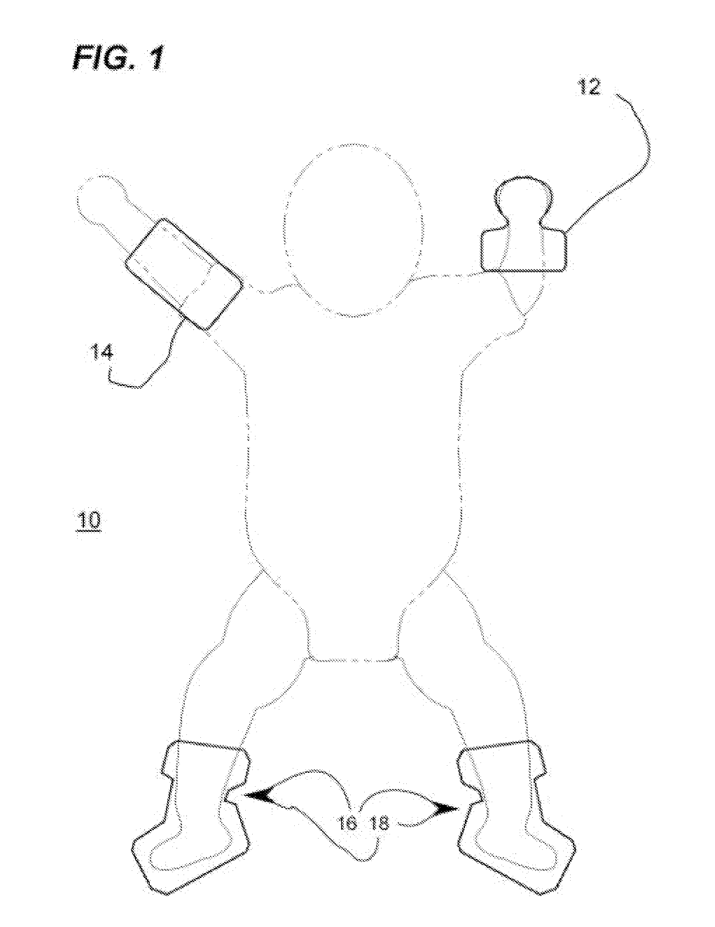

The splints (12, 14, 16) can be placed on a newborn as shown in FIG. 1. The foot splints (14, 16) can be attached on both the top and the underside of the legs or on just one side, depending upon the needs.

They are attached to the newborn using attachment techniques known in the art of splints.

As to a further discussion of the manner of usage and operation of the present invention, the same should be apparent from the above description. Accordingly, no further discussion relating to the manner of usage and operation will be provided.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

1. A neonatal set of splints comprising a hand splint; an arm splint; and a foot splint; said hand splint including a rectangular portion having two perimeter portions located at opposite end portions thereof, and a tear drop shaped portion extending integrally from a middle side portion thereof in between said two perimeter portions; said arm splint having a rectangular shape with two perimeter portions located at opposite end portions thereof; and said foot splint including two rectangular portions connected together at adjacent end portions, such that said rectangular portions form an angle to one another.

2. The set of splints of claim 1, wherein said hand splint further includes an elongated reinforcing spine member in between and parallel to said two perimeter portions and extending onto a middle section of said tear drop portion.

3. The set of splints of claim 2, wherein said arm splint further includes an elongated reinforcing spine member in between and parallel to said two perimeter portions.

4. The set of splints of claim 3, wherein one rectangular portion of said two rectangular portions of said foot splint is longer than a second of said two rectangular portions of said foot splint; and wherein said longer rectangular portion further includes an elongated reinforcing spine member along a length thereof.

5. The set of splints of claim 4, wherein each of said two rectangular portions of said foot splint has two perimeter portions located at opposite side portions thereof.

6. The set of splints of claim 1, wherein said set comprises two said hand splints; two said arm splints; and two said foot splint; wherein said rectangular portions of one of said two foot splints are connected together at an angle to one another such that said two foot splints form a mirror-image of one another and thereby form a right foot splint and a left foot splint.

7. The set of splints of claim 5, wherein each of said perimeter portions are formed from a flexible plastic material, thereby being bendable and formable, such that said respective perimeter portions are adapted to be pressed against a portion of a respective hand, arm, or leg and retain their shape.

8. The set of splints of claim 1, wherein each respective spine member is padded on an interior surface thereof.
